(19) United States
(12) Patent Application Publication
(10) Pub. No.: US 2016/0131914 A1
(43) Pub. Date: May 12, 2016

(54) HEAD-MOUNTED DISPLAY SYSTEM INCLUDING AN EYE-TRACKER SYSTEM AND MEANS FOR ADAPTATION OF THE IMAGES EMITTED

(71) Applicant: THALES, Courbevoie (FR)

(72) Inventors: Johanna LUX, Le Haillan (FR); Sebastien ELLERO, Andiran (FR); Philippe AUGEREAU, Cestas (FR)

(21) Appl. No.: 14/934,104

(22) Filed: Nov. 5, 2015

(30) Foreign Application Priority Data
Nov. 7, 2014 (FR) 1402529

(51) Int. Cl. G02B 27/01; G02B 27/30; A61B 3/113; G02B 27/00

(52) U.S. Cl. CPC G02B 27/0179; G02B 27/0172; G02B 27/0093; G02B 27/30; A61B 3/113; G02B 2027/014; G02B 2027/0185; G02B 2027/0132; G02B 2027/0181; G02B 2027/0187

(57) ABSTRACT

Visual display systems are described having means for generating stereoscopic images, a device for visual display of said stereoscopic images having a projector and a semitransparent screen; and a pair of stereoscopic spectacles. The projector according to the invention has an imager and an interference filter, the spectral transmission of which has at least one transmission band of determined width centred around a wavelength, said wavelength being dependent on the incidence of the light on said interference filter. The projector has means allowing the angular position of the filter to be varied between two determined positions so as to transmit, according to the position, either a first spectral band or a second spectral band. The pair of stereoscopic spectacles has a first lens transmitting the first spectral band and blocking the second spectral band and a second lens providing the opposite function.

The field of the invention is that of display systems carried by the head of a user. These systems enable in particular the display of information superimposed on the outside view. They are notably known as see-though head-mounted display (HMD) systems. They can be monocular or binocular. They are used in various applications. There may be cited their use in the cockpits of civil and military aircraft where they are used to present to the pilot essential information concerning piloting or navigation.

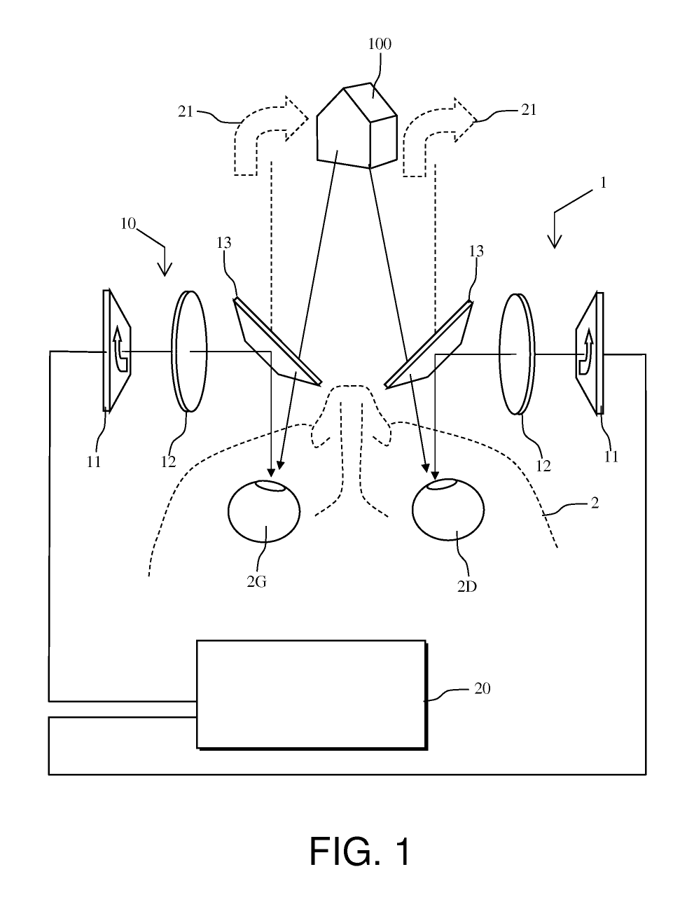

These HMD generally display virtual information at a given fixed distance from the user. This method has a number of disadvantages:
the accommodation difference between the virtual image and a real object in the outside view over which the virtual image is superimposed indicates to the user two different distances for two elements that appear superimposed;
the user cannot see these two elements clearly at the same time;
in some configurations, this situation creates a conflict of perception between accommodation and perception; accommodation indicates that the real object is close to the user and that the virtual image is farther away, whereas the masking or superimposition that is inherent in the monocular index of spatial perception indicates that the virtual image is closer than the real object since it is not hidden by the latter;
when the system is binocular, the distance difference between the virtual images and the real objects causes a well-known phenomenon known as diplopia; this problem is shown in FIGS. 1, 2 and 3 and described in detail hereinafter.

When a person looks at an object, by deformation of the crystalline lens each eye accommodates on the object to see it clearly. The two eyes converge simultaneously toward the object. The brain merges the two images coming from the left eye and the right eye in order to see only one non-duplicated object.

FIG. 1 shows a binocular HMD 1. It includes two display devices 10 disposed in front of the eyes 2G and 2D of the user 2. Each device includes a small display 11, a collimator optic 12 and an optical combiner 13 enabling an image from the display to be superimposed on the outside view. Graphic generator means 20 display the same image on both displays. In FIG. 1, the image 21 is a white curved arrow. The outside view is symbolically represented by a stylized house 100. The black arrows indicate the propagation of the light rays.

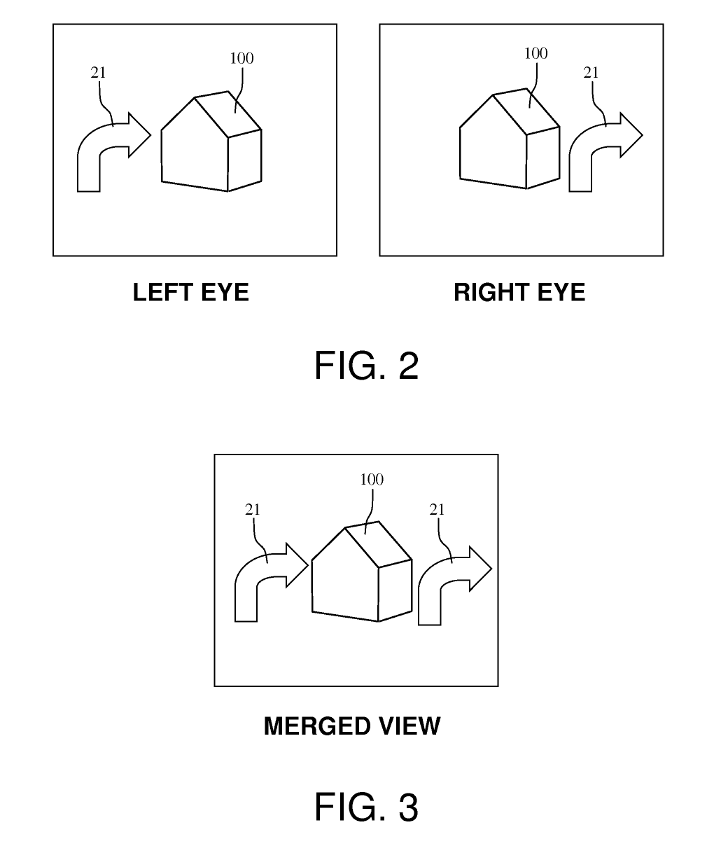

When a virtual image is presented to the user in binocular mode in the binocular HMD 1, there can be a difference between the projection distance of the image, which is conventionally at optical infinity for aeronautical applications, and the real distance of the element of the outside view onto which the displayed image is superimposed. Thus in FIG. 1 the house 100 is at a finite distance whereas the arrow is at optical infinity. As seen in FIG. 2, in this case, the left eye sees the arrow on the display to the left of the house and the right eye sees the arrow on the display to the right of the house. In merged vision, if the brain assigned priority to the outside view, the observer will see a single house and two arrows, as shown in FIG. 3. Conversely, if the brain assigns priority to the images from the displays, the observer will see a single arrow and two houses. In all cases, the real object and the virtual image cannot be seen clearly and in a non-duplicated manner at the same time.

Of course, the angular differences between the perceived images are smaller than is shown diagrammatically in FIGS. 2 and 3. In fact, the interpupillary distance in humans is approximately 65 millimetres while the distances between the user and the external objects are measured at least in metres. In aeronautics, for example, it is clear that the real objects are necessarily at a certain distance from the aircraft. However, even a small angular offset between the real image and the virtual image can eventually cause serious vision problems or serious visual fatigue, especially as the user who is not necessarily aware of it.

Different technical solutions have been proposed to overcome these different problems. The US application 2013/0088413 entitled “Method to Autofocus on Near-Eye Display” describes a display system in which the distance of the virtual image is adjustable, this distance being a function of the distance of the real objects in the outside view. This latter distance is determined either by an autofocus system as found in still cameras and video cameras or by a distance measuring system that functions by emitting optical or ultrasound signals. However, these various means for determining the distances of the various real objects of the outside view cannot tell which object the user is actually looking at.

The US application 2013/0241805 entitled “Using convergence angle to select among different UI elements” describes a display system including an eye-tracker enabling the angle of convergence between the two eyes of the user to be determined, the objects looked at to be deduced from this, and virtual images to be displayed accordingly. However, the virtual image always remains displayed at the same distance.

Thus these two technical problems do not make it possible to solve completely the problem raised by the superimposition of a virtual image on a real object.

The head-mounted display system in accordance with the invention combines and adds to the above two systems to obtain a system that simply and optimally corrects both the accommodation problem and the binocular merging problem. There is then no longer any visual perception conflict between the view and the information displayed by the display system. The observer sees clearly the HMD image or images and the outside view, with no image duplication. To be more precise, the invention consists in a head-mounted display system intended to be worn by a user, said display system being monocular and including at least one display device, an eye-tracker and graphic generator means, the display device including a display, a collimator optic and an optical combiner enabling an image from the display to be superimposed on an outside view, characterized in that:

the eye-tracker includes means for determining the angle of convergence of the two eyes of the user and the resulting accommodation;

the collimator optic includes means for projecting the image produced by the display at a distance that is a function of said accommodation;

the graphic generator means enable said image to be displayed at a position that is a function of said accommodation.

Said system is advantageously binocular and includes at least one second display device, the second display device including a second display, a second collimator optic and a second optical combiner enabling a second image from the second display to be superimposed on the outside view, the second collimator optic including means enabling the image from the second display to be projected at a distance that is a function of said accommodation and the graphic generator means enabling the second image to be displayed at a position that is a function of said accommodation.

Said display system advantageously includes a second eye-tracker, the two eye-trackers including means for determining the angle of convergence of the two eyes of the user and the resulting accommodation.

The information from the eye-tracker or eye-trackers is advantageously used to modify the parameters of the displayed image.

The information from the eye-trackers is advantageously used to monitor the vigilance of the user.

The head-mounted display system is advantageously an aeronautical system, the user being an aircraft pilot.

The invention will be better understood and other advantages will become apparent on reading the following description given by way of non-limiting example and thanks to the appended figures, in which:

FIG. 1, already commented on, represents a prior art head-mounted display system;

FIG. 2 represents the images seen by the left eye and the right eye of a user of such a system;

FIG. 3 represents the binocular view of the user of such a system;

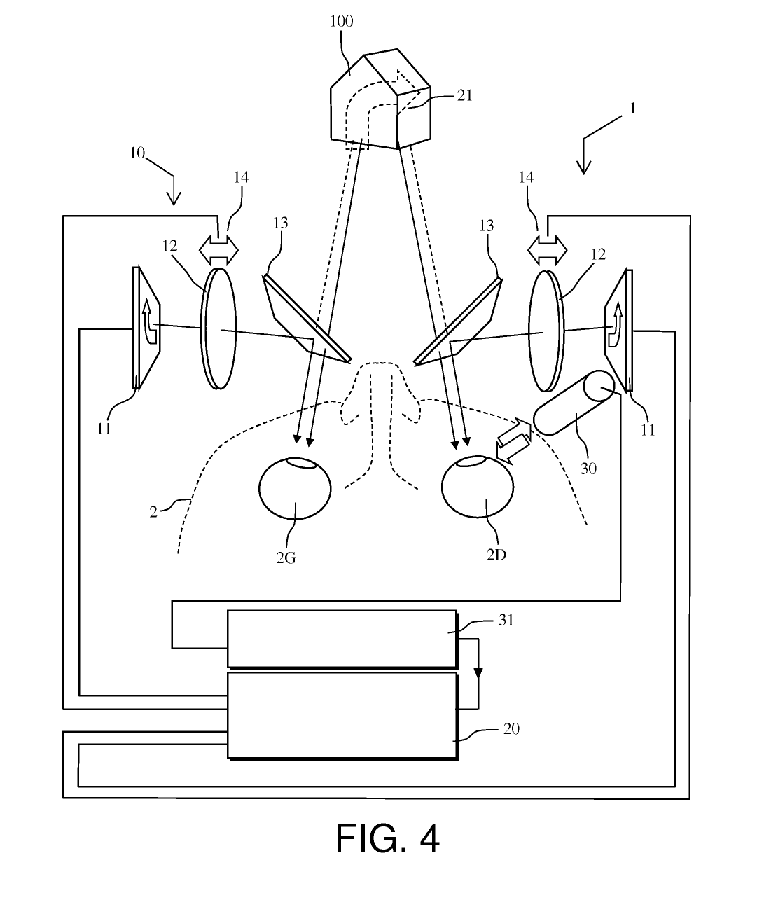

FIG. 4 represents a head-mounted display system in accordance with the invention;

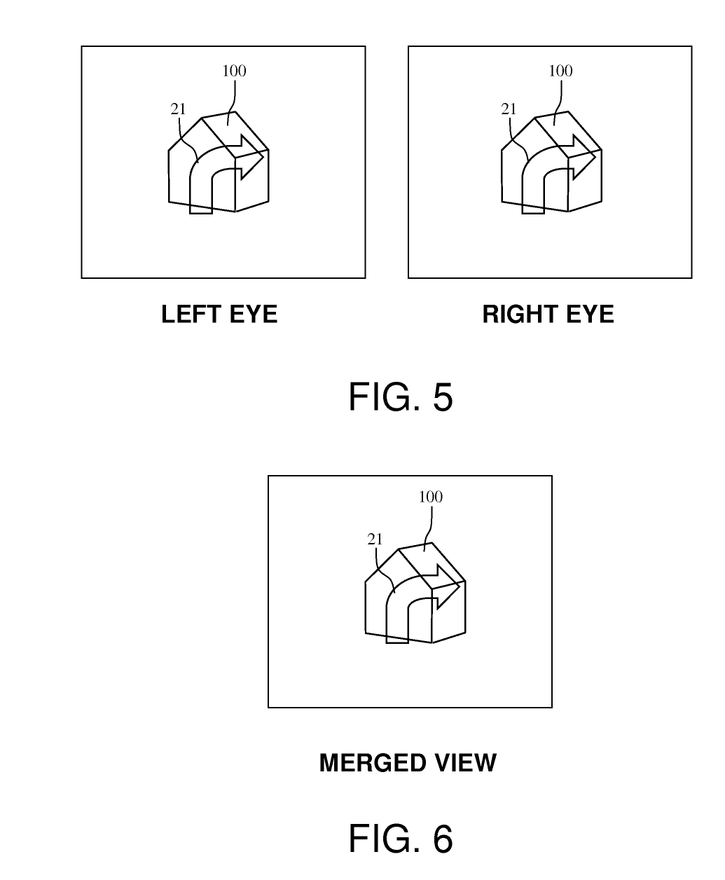

FIG. 5 represents the images seen by the left eye and the right eye of a user of such a system in accordance with the invention;

FIG. 6 represents the binocular view of a user of such a system.

The display system in accordance with the invention can be monocular or binocular. The principal application of the system in accordance with the invention is to assist with piloting aircraft. Such a system can be used for all applications necessitating the superimposition of synthetic images on the outside world. The possibility of producing head-mounted display systems at low cost makes it possible to envisage a great variety of application, as much in the transport system as in professional applications necessitating a near view or consumer applications. By way of non-limiting example, FIG. 4 represents a binocular display system. It is possible without technical difficulty to produce a monocular system based on this binocular first system.

The following description uses the same reference numbers as FIG. 1. The black arrows indicate the propagation of the light rays. The display system includes:
two display devices 10 disposed in front of the eyes 2G and 2D of the user 2; each device includes:
   a small display 11 that can be a liquid crystal display, for example;
   a collimator optic 12; in FIG. 4, the collimator optics are represented by single lenses; these optics are generally complex optical systems including a plurality of lenses or groups of lenses; in FIG. 4, the black arrows indicate the propagation of the light rays;
   an optical combiner 13 enabling an image from the display to be superimposed on the outside view; in FIG. 4, the combiner is symbolically represented by a plate with plane and parallel faces; the combiner may take other forms, have optical power or be integrated into a single visor;
   adjustment means 14 for adjusting the projection distance of the image to a particular value; these means are symbolically represented by double-headed arrows in FIG. 4; these means generally consist in moving a short distance either the display relative to the collimator optic or all or part of the collimator optic relative to the display; the means for producing this movement may be purely mechanical or electro-mechanical; it is equally possible to change an optical parameter of a lens or a mirror such as its optical index or its radius of curvature;
graphic generator means 20 display the same image on the two displays; these means enable symbols representative of navigation or piloting parameters or any other information concerning the machine to be displayed; in FIG. 4, the image 21 is a white curved arrow; the outside view is symbolically represented by a stylized house 100; these means also control the aforementioned adjustment means;
an eye-tracker 30; the function of this system is to identify the direction in which the user is looking; there exist different principles for producing this type of system; reflections on the cornea and/or the pupil of the eye are generally used to compute the convergence angle; note that the optics and the optical paths of the eye-tracker and the HMD may be in part common; thus the eye-tracker includes one or more light sources, generally in the near infrared so as not to interfere with vision, micro-camera type photoreceiver means, and analysis means 31 for computing the convergence information; that information is sent to the graphic generator means 20; note that it is possible to dispose a second eye-tracker on the other eye, either to refine the measurement or to provide redundancy.

As synergy exists between accommodation and the convergence of the eyes, it is possible to deduce the accommodation effort and therefore the distance of the observed object entirely from the measurement of the convergence angle of the eyes produced by the eye-tracker or eye-trackers. The distance between the user and the element of the outside view they are looking at is therefore known.

Knowing this accommodation and convergence information, the graphic generator means adjust simultaneously and in real time:
the two projection distances of the virtual images so that they appear at the same distance as the external element that is being looked at;
the respective positions of the images displayed on the two displays so that the projected virtual images both converge on the real object.

As seen in FIGS. 5 and 6, in this case, the left eye, the right eye and the merged view see the arrow 21 on the display perfectly superimposed on the house representing the outside view. In this case, the problems of fuzziness and image duplication have disappeared.

Using eye-trackers has other advantages. Thus the eye-tracker can serve as means for interaction with the HMD. The parameters of the displayed image can therefore be modified as a function of the knowledge of what object is being looked at. Another advantage is that the eye-tracker can serve as means for monitoring the vigilance of the user. This function can be particularly useful for all piloting applications.

It is equally possible to complement the head-mounted display system in accordance with the invention by adding a posture detector system enabling the posture of the head of the user relative to a known frame of reference to be known perfectly.

There exist various techniques for identifying an object in space. Electromagnetic detection may be used. A sender is disposed in the fixed frame of reference and a receiver in the mobile frame of reference. Passive or active optical detection may equally be used. In the latter case, the display device carries light-emitting diodes the position of the emission from which is identified by means of video cameras. All these techniques are known to the person skilled in the art. They are compatible with operation in real time and adapt easily to the display system in accordance with the invention.

1. A head-mounted display system intended to be worn by a user, said display system being monocular and comprising at least one display device, an eye-tracker and graphic generator means, the display device including a display, a collimator optic and an optical combiner enabling an image from the display to be superimposed on an outside view, wherein:
the eye-tracker includes means for determining the angle of convergence of the two eyes of the user and the resulting accommodation;
the collimator optic includes means for projecting the image produced by the display at a distance that is a function of said accommodation;
the graphic generator means enable said image to be displayed at a position that is a function of said accommodation.

2. The head-mounted display system according to claim 1, wherein said system is binocular and includes at least one second display device, the second display device including a second display, a second collimator optic and a second optical combiner enabling a second image from the second display to be superimposed on the outside view, the second collimator optic including means enabling the image from the second display to be projected at a distance that is a function of said accommodation and the graphic generator means enabling the second image to be displayed at a position that is a function of said accommodation.

3. The head-mounted display system according to claim 2, wherein said display system includes a second eye-tracker, the two eye-trackers including means for determining the angle of convergence of the two eyes of the user and the resulting accommodation.

4. The head-mounted display system according to claim 1, wherein the information from the eye-tracker or eye-trackers is used to modify the parameters of the displayed image.

5. The head-mounted display system according to claim 1, wherein the eye-trackers constitute means for monitoring the vigilance of the user.

6. The head-mounted display system according to claim 1, wherein the head-mounted display system is an aeronautical system, the user being an aircraft pilot.
